(19) United States
(12) Patent Application Publication
(10) Pub. No.: US 2025/0087315 A1
(43) Pub. Date: Mar. 13, 2025

(54) CLINICAL TRIAL OPERATIONAL PLAN GENERATION

(71) Applicant: IQVIA Inc., Parsippany, NJ (US)

(72) Inventors: Virupaxkumar Bonageri, Bangalore (IN); Rajneesh Patil, Durham, NC (US)

(21) Appl. No.: 18/885,234

(22) Filed: Sep. 13, 2024

Related U.S. Application Data
(60) Provisional application No. 63/582,390, filed on Sep. 13, 2023.

(51) Int. Cl. G16H 10/20; G06Q 10/0631; G16H 40/20

(52) U.S. Cl. CPC G16H 10/20; G06Q 10/06313; G16H 40/20

(57) ABSTRACT

Methods, systems, and apparatus, including computer programs encoded on computer storage media, for generating operational plans. The method includes grounding a natural language processing (NLP) model in clinical trial context documents containing information related to execution of clinical trials; receiving a request to generate a clinical trial operational plan; by the NLP model and responsive to the received request, generating the clinical trial operational plan such that the generated plan includes content based on the clinical trial context documents; providing the generated clinical trial operational plan as an output; receiving revisions to the generated clinical trial operational plan, wherein the revisions applied to the generated clinical trial operational plan correspond to a revised clinical trial operational plan; and grounding the NLP model in the revised clinical trial operational plan.

### CLAIM OF PRIORITY

This application claims priority to U.S. Patent Application Ser. No. 65/582,390, filed on Sep. 13, 2023, the contents of which are incorporated here by reference in their entirety.

### BACKGROUND

Clinical trials rely on operational plans to guide their execution. Accurate and complete operation plans are important to ensure the safety of clinical trial participants and the efficacy of the clinical trial.

A clinical trial or program can be a single research study or multiple research studies that prospectively assigns human participants/subjects or groups of human subjects to one or more health-related interventions to evaluate the effects on health outcomes.

### SUMMARY

The systems and techniques described here relate to generation of operational plans. By leveraging a natural language processing model and adaptive knowledge bases, users across healthcare and pharmaceutical industries can generate detailed and accurate operational plans in an efficient manner.

The proposed methods include implementing a natural language processing (NLP) model grounded in context documents and maintaining a feedback loop to continuously improve the performance of the NLP model. The NLP model provides general language and reasoning capabilities, while the feedback loop provides increasingly accurate outputs. A combination of the NLP model and the feedback loop provides users an accurate process for efficiently and consistently generating operational plans. The continuous feedback loop can consider user feedback and ensures that prompts to the NLP continuously refined to provide the most useful and relevant prompts for the specific type of operational plan that a user desires to generate.

In some cases, operational plans related to clinical trials include one or more of a protocol design, a trial participant recruitment or retention plan, a site selection or management plan, a data collection or analysis plan, or a regulatory compliance plan.

In one aspect, a method includes grounding a natural language processing (NLP) model in clinical trial context documents containing information related to execution of clinical trials, receiving, via a user interface, a request to generate a clinical trial operational plan, by the NLP model and responsive to the received request, generating the clinical trial operational plan such that the generated plan includes content based on the clinical trial context documents, providing the generated clinical trial operational plan as an output via the user interface, receiving, via the user interface, revisions to the generated clinical trial operational plan, wherein the revisions applied to the generated clinical trial operational plan correspond to a revised clinical trial operational plan; and grounding the NLP model in the revised clinical trial operational plan.

Implementations may include any or all of the following features.

In some implementations, the method further comprises generating a quality score indicative of a quality of the generated clinical trial operational plan.

In some implementations, the method comprises generating the quality score based on a comparison between the generated clinical trial operational plan and template clinical trial operational plans.

In some implementations, the method comprises generating the quality score based on a relationship between the generated clinical trial operational plan and information indicative of standard operating procedures for a clinical trial enterprise.

In some implementations, the method comprises based on the received request, generating, by a prompt engineering module, a prompt for the NLP model, and wherein the NLP model generates the clinical trial operational plan responsive to the prompt.

In some implementations, the method comprises refining operation of the prompt engineering module responsive to the received revisions to the generated clinical trial operational plan.

In some implementations, the method comprises refining operation of the prompt engineering module responsive to a quality score indicative of a quality of the generated clinical trial operational plan.

In some implementations, the method comprises anonymizing the clinical trial context documents prior to grounding the NLP model in the clinical trial context documents. In some implementations, anonymizing the clinical trial context documents comprises redacting personally identifiable information (PII). In some implementations, anonymizing the clinical trial context documents comprises redacting confidential information based on a clinical ontology.

In some implementations, the method comprises anonymizing the revised clinical trial operational plan prior to grounding the NLP model in the revised clinical trial operational plan.

In some implementations, the generated clinical trial operational plan is one or more of a protocol design, a trial participant recruitment or retention plan, a site selection or management plan, a data collection or analysis plan, or a regulatory compliance plan.

The subject matter described in this specification can be implemented in particular embodiments to realize one or more of the following advantages. Techniques are described for implementing a method for generating a clinical trial operational plans using NLP, a prompt engineering engine, and iterative feedback loops to continuously improve the quality of generated clinical trial operational plans. Users can generate clinical trial operational documents in a timely manner when faced with tight deadlines. Additionally, users can generate clinical trial operational documents that are consistently of a high quality. Generated clinical trial plans are revised until they reach a threshold quality score.

In addition to the advantages described above, the system supports multiple languages, enabling global collaboration. The system uses context documents, including high quality previously generated clinical trial operational plans, to provide guidelines for generating new clinical trial operational plans. By continuously adding to the set of context documents, the system continuously improves the quality of produced clinical trial operational plans. The system provides scalability and flexibility for multiple applications with scalable compute and storage resources to adapt to an increasing volume of data. Similarly, the system can be adapted to support multiple phases of a particular clinical trial and multiple clinical trials in different therapeutic areas. The system leverages the feedback loop and adaptive learning to tailor prompts to generate improved operational plans.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 illustrates an example approach for generating a clinical operational plan.

FIG. 2 is a flow diagram that illustrates an example process for generating a clinical operational plan.

FIG. 3 illustrates a block diagram of an example approach for generating a clinical operational plan and for refining prompts for generating a clinical operational plan.

FIG. 4 illustrates an example of generating a section of a clinical operational plan.

FIG. 5 is a flow diagram that illustrates an example process for anonymizing a generated clinical trial operational plan and generating a prompt to generate a clinical trial operational plan.

FIG. 6 illustrates a graphical user interface that displays generated text for a clinical trial operational plan and a context document.

FIG. 7 illustrates a graphical user interface that displays the generation of a clinical trial operational plan.

Like reference numbers and designations in the various drawings indicate like elements.

### DETAILED DESCRIPTION

The systems and techniques described here relate to generation of operational plans. By leveraging a natural language processing model and adaptive knowledge bases, users across healthcare and pharmaceutical industries can generate detailed and accurate operational plans in an efficient manner.

These methods include implementing a natural language processing (NLP) model grounded in context documents and maintaining a feedback loop to continuously improve the performance of the NLP model. The NLP model provides general language and reasoning capabilities, while the feedback loop enables the generation of increasingly accurate outputs. A combination of the NLP model and the feedback loop provides users an accurate process for efficiently and consistently generating operational plans. The continuous feedback loop can consider user feedback and ensures that prompts to the NLP continuously refined to provide the most useful and relevant prompts for the specific type of operational plan that a user desires to generate.

In some cases, operational plans related to clinical trials include one or more of a protocol design, a trial participant recruitment or retention plan, a site selection or management plan, a data collection or analysis plan, or a regulatory compliance plan.

Operational plans related to clinical trials can include one or more of a protocol design, a trial participant recruitment or retention plan, a site selection or management plan, a data collection or analysis plan, or a regulatory compliance plan. Multiple stakeholders within a clinical trial setting benefit from accessing complete and accurate operational plans. The stakeholders can include clinical trial participants, clinical trial site investigators, clinical trial sponsors, and regulatory authorities. For example, a particular clinical trial site investigator can use a generated clinical trial operational plan to set up a clinical trial site or maintain participant safety if a protocol is breached in order to maintain participant safety and trial integrity.

Leveraging a customized NLP model trained on operational and project plan documents and creating unique prompts based on generated operational plans improves the quality, consistency, and speed of operational plans generated by the system when compared to operational plans that are generated using manual methods. Manual methods are arduous and time consuming and require significant expertise and experience. The plans developed by manual methods are inconsistent and subjective in nature, making it difficult to measure full compliance against standard operational procedure requirements. Using he customized NLP model provides consistency in plan creation while reducing time taken in plan development.

Manual methods can result in varied quality of the resultant operational plan documents, missed details, elongated cycle times and inconsistent operational plans with inadequacies in overall details. Using the customized NLP model to create new operational plans improves protocol adherence and compliance with standard operating procedures.

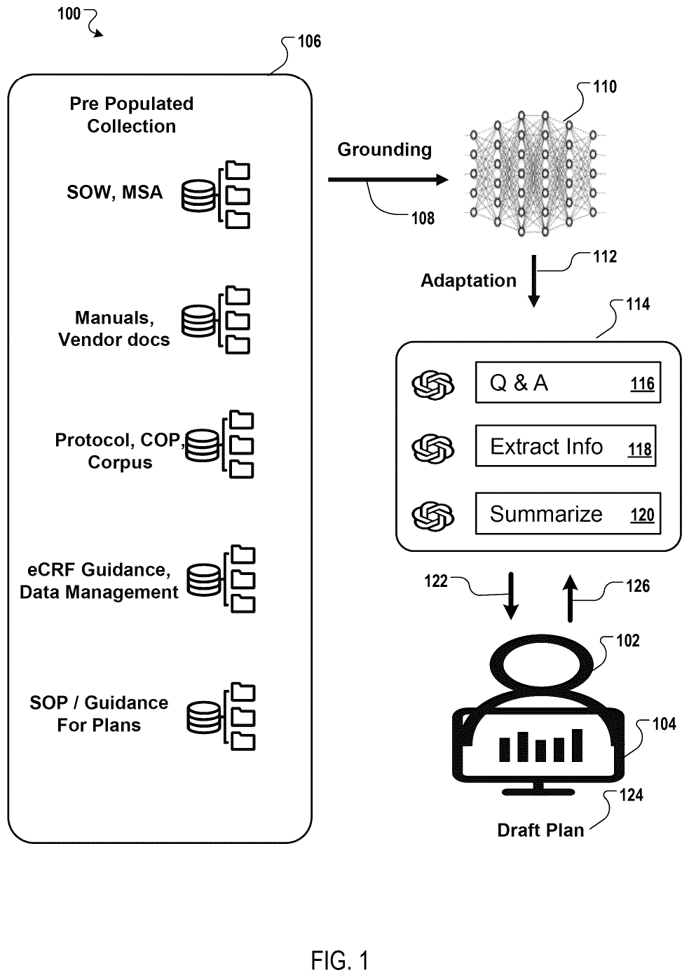

Comprehensive clinical operations planning is crucial to conducting a project (e.g., a clinical trial) in an efficient and effective manner. It allows sponsors to avoid delays in the timeline, ensure data quality (i.e., project managers can ensure consistent and complete data collection, avoiding skewed results and repeat projects), and protect participant safety (i.e., due to increased protocol adherence, monitors, and timely responses). When the operational plan accounts for all necessary steps and process, sponsors can avoid delays and budget overruns due to remedial actions. Adequate clinical operations plans address the concerns of participants during the study causing decreased dropout rates and avoids difficulties in recruiting and retaining study participants. Additionally, comprehensive clinical trial operational plans increase the chances of regulatory approval for the project. FIG. 1 illustrates an example approach 100 for generating a generating an operational plan 124. A sequence of communications between components of a system that executes operations associated with the approach 100 are denoted by a numerical indicator that is indicative of an order of communications between components. In some cases, an order of a particular communication can be changed with respect to another particular communication. In some other cases, a particular communication must be executed before another particular communication. The order of communications in relation to the approach 100 is an example order of communications and operations for generating the operational plan 124.

The system grounds 108 a natural language processing (NLP) model 110 in a set of context documents 106 containing information related to execution of a project. The project can be, for example, a clinical trial. In other examples, the project can be building an architectural structure, building a transportation system, etc.

The set of context documents 106 can include any appropriate number of documents that are relevant to the execution of the project e.g., 100 documents, 500 documents, 1000 documents, etc. In the example where the project is a clinical trial, the context documents can include, for example, protocol documents, pharmacy manuals, historical documents, lab manuals, data management guidelines, vendor documents, etc. Grounding the NLP model in specific context documents for the type of project improves the accuracy of generated operational plans compared to off the shelf models.

The NLP model 110 can be based on any suitable language model tailored to the specific project context (i.e., the context of a clinical trial) that is configured to use machine learning to generate text in a human language.

The NLP model 110 performs multiple operations including natural language processing tasks like text chunking, vector embedding, and named entity extraction. In addition, the NLP model 110 accesses one or more language models, e.g., a large language model, in which the language model processes a vector embedding of a prompt. The prompt can include a question, an instruction, or both. In some implementations, the language model includes multiple transformer neural network layers, wherein processing the prompt includes generating an embedded representation of the prompt (e.g., a numerical representation of the prompt), and processing the embedded representation of the prompt or a processed version of the embedded representation of the prompt with one or more layers of the neural network. The language model provides general language and reasoning capabilities in multiple languages. The multi-lingual capability of the system enables the system to operate globally with data sources and users of multiple languages and locations.

In order to ground 108 the NLP model 110, the system can use a grounding algorithm to connect language to the content of the context documents 106 as a part of a training process. The grounding algorithm can be, for example, a spectral algorithm that vectorizes the documents.

The system can receive, via a user interface 104 a request to generate an operational plan for completing a project.

A user 102 accesses a user interface 104. The user can be, for example, a clinical investigator. In some implementations, the user interface 104 is implemented on a computing device that is connected to the internet. In some implementations, the user interface 104 is implemented on a computing device, in which the computing device also implements operations according to one or more other components of the system including the NLP model 110 and a prompt engineering module 114. In some implementations, one or more of the components of the system are remote in relation to the other components of the system and are communicatively coupled to at least one other component of the system.

In some implementations, the user 102 enters a request using the user interface 104. The computing device that hosts the user interface 104 can transmit the request to the NLP model 110. The NLP model 110 processes a vector embedding of a prompt that includes at least a representation of the request.

When the project is a clinical trial, example requests include, “Generate a clinical trial operational plan for [medication name].” and “Identify a clinical trial operational plan for a clinical trial that investigates interactions between [medication A] and [medication B]”. A clinical trial operational plan can be one or more of a protocol design, a trial participant recruitment or retention plan, a site selection or management plan, a data collection or analysis plan, or a regulatory compliance plan.

A clinical trial operational plan can include multiple sections. For example, a clinical operational plan can include sections for each of protocol deviation, subject selection, project communication, drug management, site identification, quality management, financial managements, risk management, etc. A clinical trial operational plan can include a predetermined list of sections in a particular order. The list of sections can be any appropriate number of sections e.g., 10 sections, 25 sections, 50 sections, etc. each section can have one or more sub sections.

The system adapts 112 the request using the prompt engineering module 114 to generate prompts for the NLP model 110. When the project is a clinical trial, the prompt engineering module 114 can generate one or more prompts for each section of a clinical trial operational plan. Example prompts include “How do we manage protocol deviation for a clinical trial on [medication name]?” and “What should we do if the [medication name] has been managed in an improper environment”.

For a particular section, the prompt engineering module 114 can generate a sequence of multiple prompts to generate a particular section of the operational document, where each prompt depends on the output of the previous prompt. For example, the prompt engineering module can generate a prompt to query the NLP module to find all information relevant to a particular section of a operational plan 116, for example, “Find guidelines of handling mismanagement of a drug that is being investigated in a clinical trial”. The prompt engineering module can then generate a follow up prompt to extract the information relevant to a particular situation 118, for example, “Identify information regarding handling a drug that is stored in an improper temperature”. The prompt engineering module can then generate a prompt to summarize the extracted information 120, for example “Summarize the procedures for handling a drug that is stored in an improper temperature”.

The prompt engineering module 114 can process the request to generate prompts in a format that is suitable to query the NLP model 110. For example, the prompt engineering module can create an embedded representation of the request, a tokenized representation of the request, or any other suitable modification of the request.

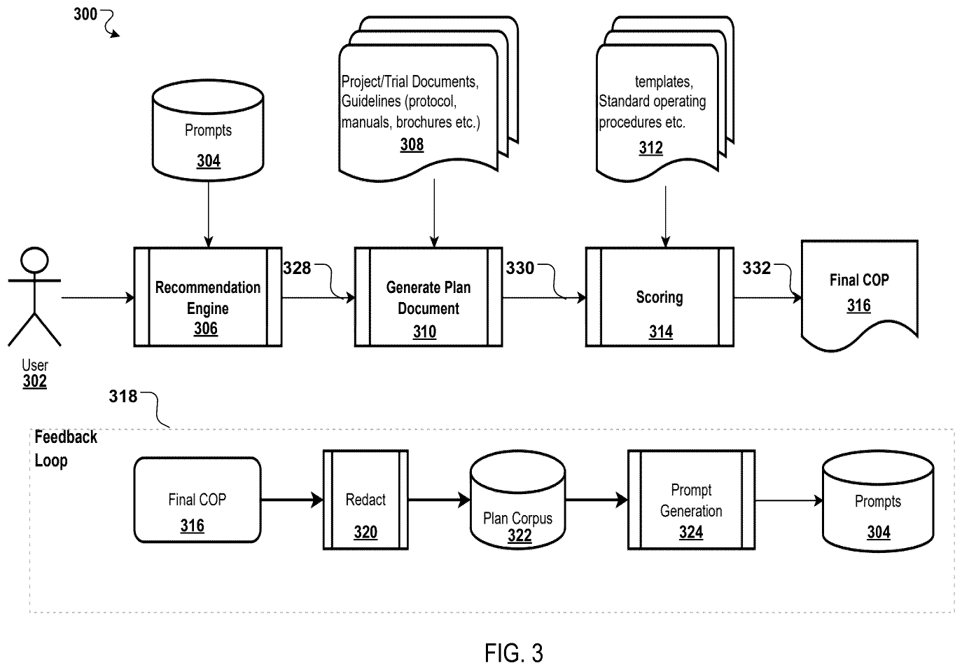

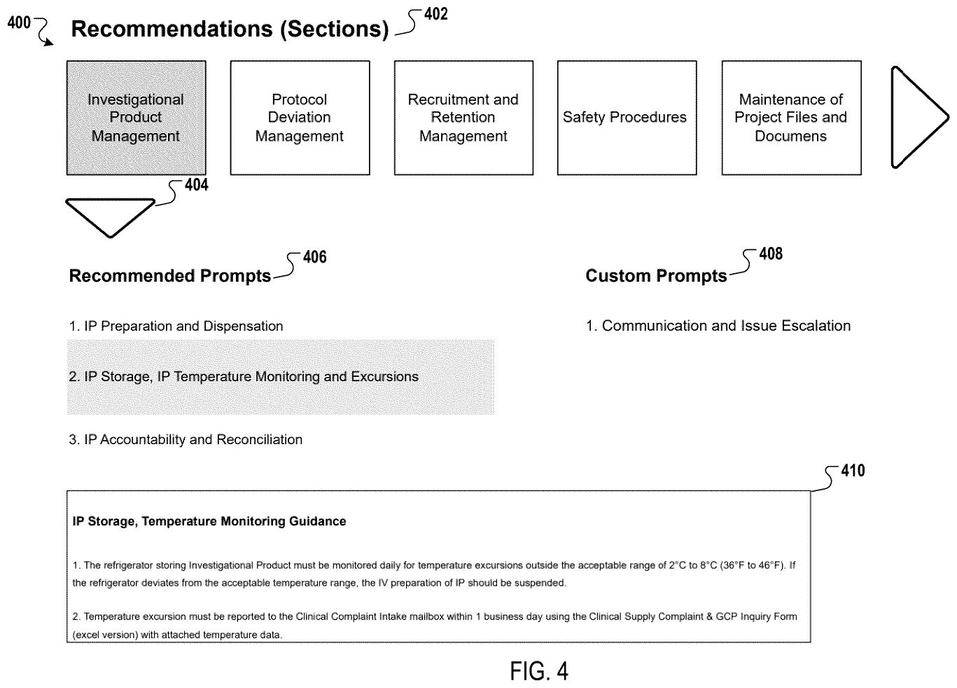

Generating prompts for the NLP model will be described in further detail below with reference to FIG. 3 and FIG. 4.

In response to the received request, the NLP model generates the operational plan 124 such that the generated plan includes content based on the context documents. The operational plan can follow a predetermined format derived from the context documents. The NLP model can compile the responses to each prompt into a single document in a predetermined order.

An output operational plan 124, in which the output operational plan 124 is a processed or unprocessed output of the NLP model 110, is transmitted to the user interface 104 to be displayed to the user 102. In some implementations, the output operational plan 124 is a text-based response to the request. In some implementations, the output operational plan 124 includes a text-based response to the request and details of the context documents 106 used to generate the operational plan. Details of the context documents 106 can include relevant sections of each context document, links to the context documents, summaries of the context documents, and/or other derived representations of the context documents.

The system can generate a quality score that evaluates the quality of the generated operational plan 124. In some examples, the quality score can be generated based on a comparison between the generated operational plan and a template operational plan of the same type. In other examples, the quality score can be generated based on a relationship between the generated operational plan 124 and information indicative of standard operating procedures for an enterprise.

The quality score can be generated using a retrieval-augmented generation (RAG) technique. The RAG technique can use any combination of context documents, template operational plans, and standard operating procedures as a knowledge repository to compare the generated operational plan 124 with. The quality score can measure both the quality of the and the quality of the content structure of the generated operational plan 124. The quality score is highest when all expected information for the particular type of operational plan is included in the generated operational plan. The quality score decreases as the amount of missing information increases.

If the quality score is below a predetermined threshold, the system receives instructions to revise the generated operational plan via the user interface 104. A user can provide revisions 126 to the generated operational plan via the user interface 104. The revisions applied to the generated operational plan correspond to a revised operational plan. The system can calculate a quality score for the revised operational plan. The user 102 can provide revisions via the user interface 104 until the generated operational plan exceeds a predetermined quality score.

The system then grounds the NLP model in the revised clinical trial operational plan. This allows the NLP model to gain more context for generation of future operational plans.

In some implementations, the system continuously monitors the context documents 106 to maintain accuracy and relevance. The system updates the context documents based on the accuracy and relevance of the reference documents, in which the accuracy and relevance of the reference documents can be based on new research findings, user feedback, and emerging trends. In some cases, the system employs natural language processing (NLP) approaches to analyze new documents, findings, and research to determine if new reference documents are added to the set of context documents 106.

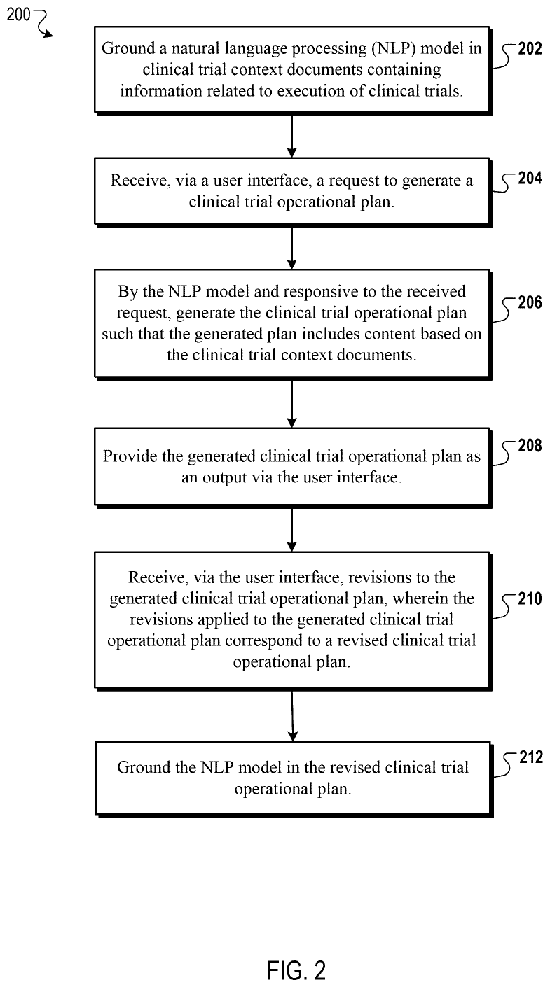

FIG. 2 is a flow diagram that illustrates an example of a process 200 for generating a operational plan. The process can be performed by a system similar to the system described in relation to the operations of the approach 100, which can include one or more computer systems. For convenience, the system is described in the context of generating a clinical trial operational plan. However, a system can perform the process 200 to generate any type of operational plan.

The system grounds (202) a natural language processing (NLP) model in clinical trial context documents containing information related to execution of clinical trials. The context documents can include, for example, protocol documents, pharmacy manuals, historical documents, lab manuals, data management guidelines, vendor documents, etc. The NLP model can be any appropriate type of language model. In most cases, an input of a language model is an embedded, e.g., vectorized or encoded, representation of a prompt. An embedded representation of the prompt is a numerical representation of the prompt in a vector space defined by parameters of the language model.

In some cases, the system anonymizes the clinical trial context documents prior to grounding the NLP model in the clinical trial context documents, e.g., to remove personally identifiable information, commercially sensitive information (e.g., proprietary terminology or data), or other sensitive information.

The system receives (204) a request to generate a clinical trial operational plan. In some implementations, the system receives the request from a user that accesses a user interface. The user interface can include a chat interface, or any text or speech interface that can capture an input from a user. In some cases, the request is an instruction to generate a clinical trial operational plan. The clinical trial operational plan can be one or more of a protocol design, a trial participant recruitment or retention plan, a site selection or management plan, a data collection or analysis plan, or a regulatory compliance plan.

In some cases, a prompt engineering engine can generate one or more prompts to be processed by the NLP model from the request using a prompt engineering module. In some implementations, the one or more prompts include a single prompt. In some other implementations, the one or more prompts include a sequence of prompts, in which each prompt of the sequence of prompts uses an output of a language model corresponding to a preceding prompt as part of a subsequent prompt.

In some implementations, the system includes instructions and/or style guidelines as part of the prompt. For example, the system can include statements like, “Consider the following context documents: [document content]” as part of the prompt to ensure the language model uses the provided context document as the source of information for a response.

The system generates (206) a clinical trial operational plan using the NLP model in response to the received request. The generated plan includes content based on the clinical trial context documents. In some cases, the NLP model generates the clinical trial operational plan by processing a sequence of prompts and compiling some or all of the outputs of the NLP model.

The system provides (208) the generated clinical trial operational plan as an output. The system can provide the clinical trial operational plan to a user via a user interface. In some cases, the system embeds one or more prompts and processes the embedded prompts with multiple layers of a neural network to produce an output embedding that represents the output of the NLP model. In some cases, the output is a response to the request, e.g., a clinical trial operational plan. In some cases, the output includes the response to the request and relevant context documents used to provide context for generating the response, e.g., the clinical trial operational plan. The system can provide relevant context documents in the form of full-text documents, links to documents, relevant sub-sections of documents, AI-generated summaries of the documents, etc.

In some cases, the system can evaluate the generated clinical trial operational plan using a quality score. In some examples, the system can generate the quality score based on a comparison between the generated clinical trial operational plan and a template operational plan of the same type. In other examples, the system can generate the quality score based on a relationship between the generated operational plan and information indicative of standard operating procedures for a clinical enterprise.

If the quality score is lower than a predetermined threshold, the system can revise the generated clinical trial operational plan.

The system receives (210) revisions to the generated clinical trial operational plan. The system can receive the revisions via a user interface. The revisions applied to the generated clinical trial operational plan correspond to a revised clinical trial operational plan. The system can assess the revised clinical trial operational plan based on a quality score. If the revised clinical trial operational plan receives a quality score that is below a predetermined threshold, the system can receive additional revisions to the clinical trial operational plan.

The system grounds (212) the NLP model in the revised clinical trial operational plan. This allows the system to use the revised clinical trial operational plan in the generation of additional clinical trial operational plans. This improves the quality and consistency of future clinical trial operational plans.

In some cases, the system refines the operation of a prompt engineering module in response to the received revisions to the generated clinical trial operational plan. In some cases, the system can refine the operation of the prompt engineering module in response to a quality score indicative of a quality of the generated clinical trial operational plan.

In some cases, the system can receive a clinical trial operational plan generated from a source that is not the NLP model. The system can score the received plan by generating a quality score indicative of a quality of the received clinical trial operational plan. Generating the quality score can include comparing the received clinical trial operational plan with template clinical trial operational plans, information indicative of standard operating procedures for a clinical trial enterprise, or both. Similarly to the case where the system generates a clinical trial operational plan, the system can receive revisions to the received clinical trial operational plan. The system can update the received clinical trial operational plan based on the generated quality score and the received revisions to produce a revised clinical trial operational plan. Once the system revises the clinical trial operational plan, the system can ground a NLP model in the revised clinical trial operational plan. The NLP model can be previously grounded in clinical trial context documents containing information related to execution of clinical trials and configured to generate clinical trial operational plans responsive to prompts. The context documents can include clinical trial operational plans that were previously generated by the NLP.

FIG. 3 illustrates a block diagram of an example approach for generating a clinical operational plan and for refining prompts for generating a clinical operational plan. The approach 300 includes operations performed by components of a system, in which the system includes a recommendation engine 306, a natural language processing (NLP) model 310, a scoring module 314, and a feedback loop 318. The approach 300 includes multiple steps of a process for generating a clinical trial operational plan 316 in response to a user request 302 that are executed on at least one computing device. In some implementations, a single computing device executes the steps of the process. In some other implementations, multiple computing devices that are communicatively coupled execute the steps of the process.

The user request 302 is an instruction to generate a clinical trial operational plan for a particular clinical trial. In some implementations, the clinical trial operational plan 316 is displayed to the user by a user interface or other means of communicating a response to the user. In some implementations, the user interface that collects the user request 302 is the same user interface that displays the clinical trial operational plan 316. In some other implementations, two distinct user interfaces are employed.

The recommendation engine 306 receives the user request 302 and generates a set of recommended relevant prompts 328 from a prompt database 304 to be processed by the NLP model. The recommended prompts 328 can be, for example, a set of default prompts for building a clinical trial operational plan. In some cases, the recommended prompts 328 can be all prompts in the prompts database 304 that mention a particular medication that is being evaluated in a clinical trial.

The NLP model 310 is grounded in a set of context documents 308. The context documents 308 can include project protocol, project guidelines, manuals, brochures, etc. The NLP model processes the recommended prompts to generate an initial clinical trial operational plan document 330.

When the NLP model is a large language model, multiple layers of a neural network that define the large language model process an embedded representation of each prompt to output the initial clinical trial operational plan document 330. In the context of large language models and GPTs, the processing of prompts to generate an output is referred to as text completion. The output of the large language model is received by the user by a user interface.

The scoring module 314 receives the initial clinical trial operational plan document 330 and generates a quality score 332 for the initial clinical trial operational plan document using a scoring algorithm (e.g., a cosine similarity algorithm) that compares the generated clinical trial operational plan document to an example document from a set of example documents 312. In some cases, the quality score 332 can be based on a comparison between the generated clinical trial operational plan 330 and template clinical trial operational plans. In some cases, the quality score 332 can be based on a relationship between the generated clinical trial operational plan 330 and information indicative of standard operating procedures for a clinical trial enterprise. The quality score indicates the quality of the generated clinical trial operational plan.

If the quality score 332 is lower than a predetermined threshold, the system can perform corrective actions to the initial clinical trial operational plan based on the example documents 312 to generate a revised clinical trial operational plan 316. The system provides the revised clinical trial operational plan 316 to a feedback loop.

The feedback loop 318 includes a redaction engine 320 and a prompt engineering module 324. The redaction engine 320 receives the revised clinical trial operational plan 316 anonymizes the revised clinical trial operational plan. The redaction engine can redact personally identifiable information e.g., redacting email ids and protocol numbers. The redaction engine can also redact confidential information based on a clinical ontology e.g., redacting new drug names and other scientific information.

The redaction engine 320 provides the anonymized clinical trial operational plan to a set of operational plan documents 322 that have all been anonymized. The operational plan documents include previously generated clinical trial operational plans that exceed the predetermined quality score.

The prompt engineering module uses the set of operational plan documents 322 to add new prompts to the prompts database 304. The prompt engineering module can generate prompts that result in generating clinical trial operational plans with higher quality scores in response to the revised clinical trial operational plan 316. If the quality score of a generated clinical trial operational plan is below a predetermined threshold, the prompt engineering engine can refine prompts that can generate a clinical trial operational plans with higher quality scores.

Refining the prompts based on the revised clinical trial operational plan 316 allows the system to recommend prompts that fill in information that was missing in the initial clinical trial operational plan document 330. Future plans generated by the system will include the missing information before revisions, resulting in higher quality scores for new generated clinical trial operational plans. The system can provide, via a user interface, potential sections and information that are missing from the initial clinical trial operational plan to a user. For each potential missing section or piece of information, the user can chose to either prompt the NLP to generate each potential missing section or piece of information or ignore the potential missing section or piece of information.

Every time that a new clinical trial operational plan is generated, the system can use the clinical trial operational plan as context to improve the quality of future clinical trial operational plans generated by the same system. The system can recommend more accurate prompts for generating operational plans that require fewer iterations of revisions.

FIG. 4 illustrates an example 400 of generating a section of a clinical operational plan. The example 400 can be performed by components of a system, in which the system includes computing devices, databases, and user interfaces. In some implementations, a single computing device performs the operations. In some other implementations, multiple computing devices that are communicatively coupled perform the operations.

The example shows recommended sections 402 for the generation of a clinical trial operational plan. A recommendation engine, such as the recommendation engine 306 of FIG. 3, can provide the recommended sections 402. The recommended sections include “Investigational Product (IP) Management”, “Protocol Deviation Management”, “Recruitment and Retention Management”, “Safety Procedures”, and “Maintenance of Project Files and Documents”.

For the section “IP Management”, the example shows recommended prompts for the section 406, a generated section 410 of an operational plan, and a custom prompt 408 to add missing information to the operational plan.

The recommendation engine can generate the recommended prompts 406 for the section. The recommended prompts 406 for the “IP Management” section include “IP preparation and Dispensation”, “IP Storage, IP Temperature Monitoring and Excursions”, and “IP Accountability and Reconciliation”.

A NLP model, such as the NLP model 306 of FIG. 3, can process the prompts to generate the generated section 410 of the operational plan. The example shows the generated section for the “IP Storage, IP Temperature Monitoring and Excursions” prompt, which reads:
“1. The refrigerator storing investigational product must be monitored daily for temperature excursions outside the acceptable range of 2° C. to 8° C. (36° F. to 46° F.). If the refrigerator deviates from the acceptable temperature range, the IV preparation of IP should be suspended.
2. Temperature excursion must be reported to the Clinical Complaint Intake mailbox within 1 business day using the Clinical Supply Intake & GCP Inquiry Form (excel version) with the attached temperature data”.

A scoring module, such as the scoring module 314 of FIG. 3, can evaluate the generated operational plan to indicate if more prompts should be submitted to the NLP model. A user can provide or prompt engineering engine, such as the prompt engineering engine 324 of FIG. 3, can generate one or more custom prompts 408 to mitigate deficiencies in generated operational plans.

For example, if a scoring module generates a quality score that indicates that the quality of the generated operational plan is below a predetermined threshold, a user or a prompt engineering engine can provide one or more custom prompts that can improve the quality score.

The example shows a custom prompt 408 “Communication and Issue Escalation”. This custom prompt is a prompt to include missing information that was not generated solely based on the recommended prompts.

For generation of future operational plans, the system can use a feedback loop to incorporate the custom prompt 408 as a recommended prompt 406, increasing the quality of future operational plans.

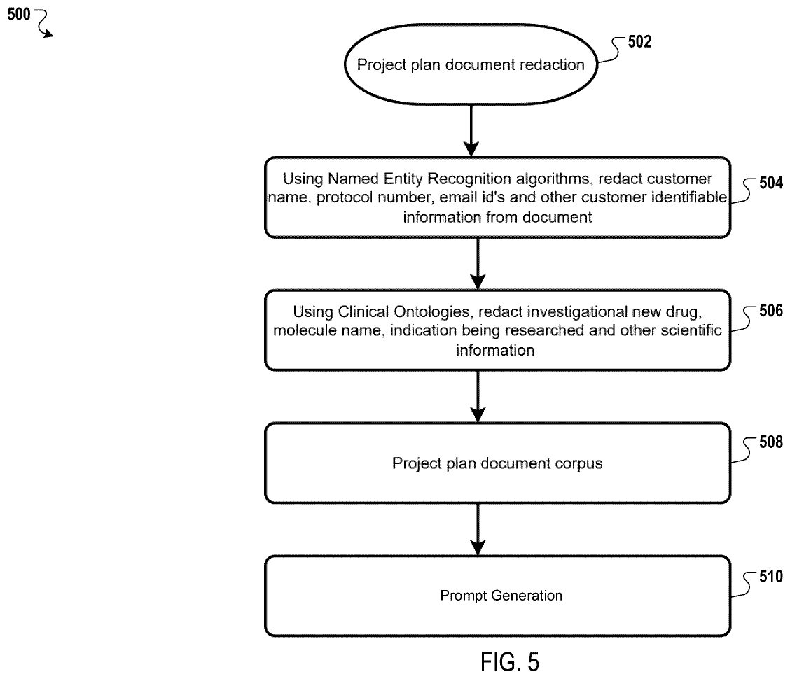

FIG. 5 is a flow diagram that illustrates an example process 500 for anonymizing a generated clinical trial operational plan and generating a prompt to generate a clinical trial operational plan. The process can be performed by a system similar to the system described in relation to the operations of the approach 300, which can include one or more computer systems.

The system redacts (502) sensitive information from documents used to ground a NLP model. In some cases, the system also redacts sensitive information from context documents prior to grounding the NLP model in the context documents. The system can redact sensitive information from a generated or revised clinical trial operational plan before grounding the NLP model in the clinical trial operational plan. This ensures that sensitive information is secure.

The redacting includes redacting personally identifiable information from the revised operational plan or context documents (504). The system can use a Named Entity Recognition algorithm to redact the personally identifiable information. The personally identifiable information can include names, protocol numbers, email IDs, and other information that can identify a person involved in the project.

The redacting also includes redacting confidential information based on a clinical ontology (506). The confidential information can include the names of investigational new drugs, molecule names, indication being researched, and other scientific information.

The system can add (508) the anonymized documents to a set of anonymized documents including clinical trial operational plans, context documents, or both.

The system can generate (510) prompts for the NLP model to process using the anonymized operational plans and context documents.

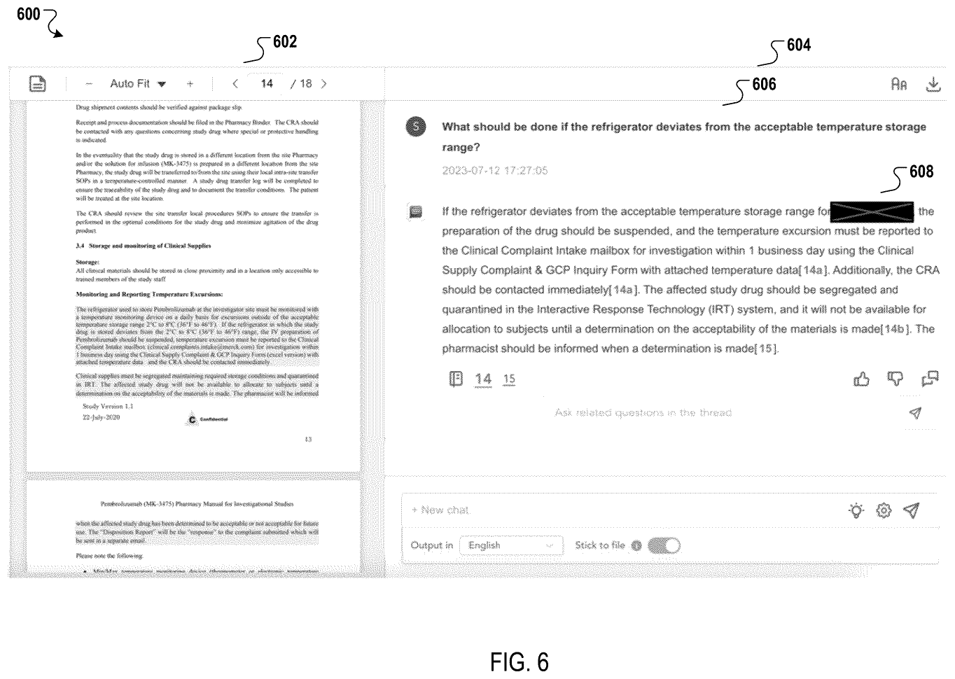

FIG. 6 illustrates a graphical user interface that displays generated text for a clinical trial operational plan and a context document.

The left pane 604 of the user interface displays a prompt 606 that reads “What should be done if the refrigerator deviates from the acceptable temperature storage temperature range?” and an associated text response 608 generated by a NLP model to respond to the prompt.

The right pane 602 of the user interface shows a highlighted section of a context document used to generate the text.

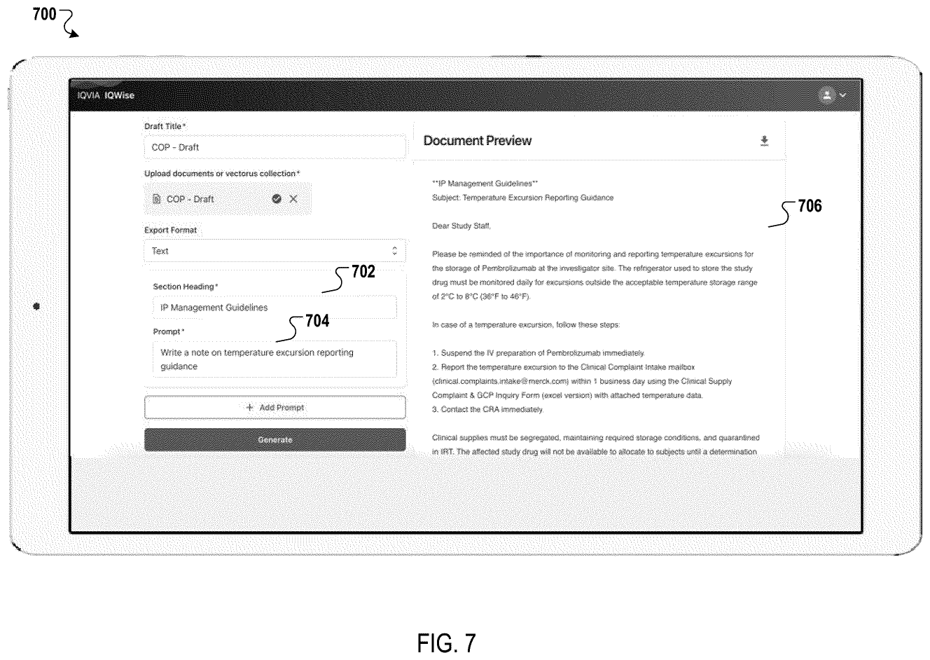

FIG. 7 illustrates a graphical user interface that displays the generation of a clinical trial operational plan.

A user can input a section heading 702 and a prompt 704 to the user interface. The user can input a sequence of section headings and one or more prompts per section heading to generate a clinical trial operational plan.

The user interface shows the progress of the generation of the clinical trial operational plan 706. The user can review and edit the generated draft plan prior to finalization.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of, data processing apparatus. Alternatively, or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. The computer storage medium can be a machine-readable storage device, a machine-readable storage substrate, a random or serial access memory device, or a combination of one or more of them. The computer storage medium is not, however, a propagated signal.

The term “data processing apparatus” encompasses all kinds of apparatus, devices, and machines for processing data, including by way of example a programmable processor, a computer, or multiple processors or computers. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

Computers suitable for the execution of a computer program include, by way of example, can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

While this specification contains specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A computer implemented method comprising:
grounding a natural language processing (NLP) model in clinical trial context documents containing information related to execution of clinical trials;
receiving, via a user interface, a request to generate a clinical trial operational plan;
by the NLP model and responsive to the received request, generating the clinical trial operational plan such that the generated plan includes content based on the clinical trial context documents;
providing the generated clinical trial operational plan as an output via the user interface;
receiving, via the user interface, revisions to the generated clinical trial operational plan, wherein the revisions applied to the generated clinical trial operational plan correspond to a revised clinical trial operational plan; and
grounding the NLP model in the revised clinical trial operational plan.

2. The method of claim 1, further comprising generating a quality score indicative of a quality of the generated clinical trial operational plan.

3. The method of claim 2, comprising generating the quality score based on a comparison between the generated clinical trial operational plan and template clinical trial operational plans.

4. The method of claim 2, comprising generating the quality score based on a relationship between the generated clinical trial operational plan and information indicative of standard operating procedures for a clinical trial enterprise.

5. The method of claim 1, comprising based on the received request, generating, by a prompt engineering module, a prompt for the NLP model, and wherein the NLP model generates the clinical trial operational plan responsive to the prompt.

6. The method of claim 5, comprising refining operation of the prompt engineering module responsive to the received revisions to the generated clinical trial operational plan.

7. The method of claim 5, comprising refining operation of the prompt engineering module responsive to a quality score indicative of a quality of the generated clinical trial operational plan.

8. The method of claim 1, comprising anonymizing the clinical trial context documents prior to grounding the NLP model in the clinical trial context documents.

9. The method of claim 8, wherein anonymizing the clinical trial context documents comprises redacting personally identifiable information (PII).

10. The method of claim 8, wherein anonymizing the clinical trial context documents comprises redacting confidential information based on a clinical ontology.

11. The method of claim 1, comprising anonymizing the revised clinical trial operational plan prior to grounding the NLP model in the revised clinical trial operational plan.

12. The method of claim 1, wherein the generated clinical trial operational plan is one or more of a protocol design, a trial participant recruitment or retention plan, a site selection or management plan, a data collection or analysis plan, or a regulatory compliance plan.

13. A system for predicting healthcare-specific responses to queries, the system comprising:
at least one processor; and
a memory storing instructions that, when executed by the at least one processor, cause the at least one processor to perform operations comprising:
grounding a natural language processing (NLP) model in clinical trial context documents containing information related to execution of clinical trials;
receiving, via a user interface, a request to generate a clinical trial operational plan;
by the NLP model and responsive to the received request, generating the clinical trial operational plan such that the generated plan includes content based on the clinical trial context documents;
providing the generated clinical trial operational plan as an output via the user interface;
receiving, via the user interface, revisions to the generated clinical trial operational plan, wherein the revisions applied to the generated clinical trial operational plan correspond to a revised clinical trial operational plan; and
grounding the NLP model in the revised clinical trial operational plan.

14. The system of claim 13, the operations further comprising generating a quality score indicative of a quality of the generated clinical trial operational plan.

15. The system of claim 14, the operations comprising generating the quality score based on a comparison between the generated clinical trial operational plan and template clinical trial operational plans.

16. The system of claim 14, the operations comprising generating the quality score based on a relationship between the generated clinical trial operational plan and information indicative of standard operating procedures for a clinical trial enterprise.

17. The system of claim 13, the operations comprising based on the received request, generating, by a prompt engineering module, a prompt for the NLP model, and wherein the NLP model generates the clinical trial operational plan responsive to the prompt.

18. The system of claim 17, the operations comprising refining operation of the prompt engineering module responsive to the received revisions to the generated clinical trial operational plan.

19. The system of claim 17, the operations comprising refining operation of the prompt engineering module responsive to a quality score indicative of a quality of the generated clinical trial operational plan.

20. One or more non-transitory computer readable media storing instructions that, when executed by at least one processor, cause the at least one processor to predicting healthcare-specific responses to queries by performing operations comprising:
grounding a natural language processing (NLP) model in clinical trial context documents containing information related to execution of clinical trials;
receiving, via a user interface, a request to generate a clinical trial operational plan;
by the NLP model and responsive to the received request, generating the clinical trial operational plan such that the generated plan includes content based on the clinical trial context documents;
providing the generated clinical trial operational plan as an output via the user interface;
receiving, via the user interface, revisions to the generated clinical trial operational plan, wherein the revisions applied to the generated clinical trial operational plan correspond to a revised clinical trial operational plan; and
grounding the NLP model in the revised clinical trial operational plan.

21. The non-transitory computer readable media of claim 20, the operations further comprising generating a quality score indicative of a quality of the generated clinical trial operational plan.

22. The non-transitory computer readable media of claim 21, the operations comprising generating the quality score based on a comparison between the generated clinical trial operational plan and template clinical trial operational plans.

23. The non-transitory computer readable media of claim 21, the operations comprising generating the quality score based on a relationship between the generated clinical trial operational plan and information indicative of standard operating procedures for a clinical trial enterprise.

24. The non-transitory computer readable media of claim 20, the operations comprising based on the received request, generating, by a prompt engineering module, a prompt for the NLP model, and wherein the NLP model generates the clinical trial operational plan responsive to the prompt.

25. A computer implemented method comprising:
receiving a clinical trial operational plan;
generating a quality score indicative of a quality of the received clinical trial operational plan, including comparing the received clinical trial operational plan with template clinical trial operational plans, information indicative of standard operating procedures for a clinical trial enterprise, or both;
receiving, via a user interface, revisions to the received clinical trial operational plan;
updating the received clinical trial operational plan based on the generated quality score and the received revisions to produce a revised clinical trial operational plan; and
grounding a natural language processing (NLP) model in the revised clinical trial operational plan, the NLP model having been previously grounded in clinical trial context documents containing information related to execution of clinical trials and configured to generate clinical trial operational plans responsive to prompts.
